(19) United States
(12) Patent Application Publication
(10) Pub. No.: US 2005/0027371 A1
(43) Pub. Date: Feb. 3, 2005

(54) Artificial limb with relative position-adjustable upper and lower limb parts

(76) Inventor: Sen-Jung Chen, Taipei City (TW)

Correspondence Address: LADAS & PARRY, 5670 WILSHIRE BOULEVARD, SUITE 2100, LOS ANGELES, CA, 90036-5679, US

(21) Appl. No.: 10/632,626

(22) Filed: Aug. 1, 2003

(51) Int. Cl. A61F 2/62

(52) U.S. Cl. 623/38

(57) ABSTRACT

An artificial limb includes a prosthetic upper limb part, a prosthetic lower limb part, an upper connecting piece disposed under and connected fixedly to the upper limb part, a lower connecting piece disposed under the upper connecting piece and disposed over and connected fixedly to the lower limb part, and a locking member for locking the upper and lower connecting pieces releaseably on each other. The locking member is operable to permit relative movement of the upper and lower connecting pieces in a transverse direction of the artificial limb in such a manner that the upper and lower connecting pieces are retained on the locking member.

### BACKGROUND OF THE INVENTION

1. Field of the Invention

This invention relates to an artificial limb, and more particularly to an artificial limb that includes upper and lower limb parts, the relative position of the upper and lower limb parts being adjustable.

2. Description of the Related Art

Referring to FIG. 1, a conventional artificial limb is shown to include an upper limb part 5, a lower limb part 6, and a connecting piece 4. The connecting piece 4 has a top end 41 that is formed with an integral tongue 411, and a hollow circular lower end portion 42 that is sleeved around a cylindrical upper end 61 of the lower limb part 6. The tongue 411 is inserted into a retaining hole 50 that is formed in a bottom surface of the upper limb part 5. Four inclined lock bolts 51 extend respectively through four bolt holes 52 in a surrounding wall 53 defining the retaining hole 50, press against the tongue 411 of the connecting piece 4, and engage an annular groove 412 in the tongue 411 so as to lock the connecting piece 4 on the upper limb part 5. The lower end portion 42 of the connecting piece 4 has a vertical slot 421, first and second projections 43, 44, and a horizontal lock bolt 45. The first and second projections 43, 44 are located at two sides of the slot 421, and are adjacent to the slot 421. The horizontal lock bolt 45 extends through a horizontal non-threaded hole 431 in the first projection 43, and engages a horizontal threaded hole 441 in the second projection 44. The horizontal lock bolt 45 is screwed tight to lock the lower limb part 6 on the connecting piece 4. When the horizontal lock bolt 45 is loosened, the lower limb part 6 can be removed from the connecting piece 4. As the upper limb part 5 is fixed relative to the lower limb part 6, the amputee may fall easily due to loss of balance during walking.

### SUMMARY OF THE INVENTION

The object of this invention is to provide an artificial limb that includes upper and lower limb parts which are interconnected by upper and lower connecting pieces so that relative position of the upper and lower limb parts can be adjusted to maintain the balance of the amputee when the amputee walks.

According to this invention, an artificial limb includes a prosthetic upper limb part, a prosthetic lower limb part, an upper connecting piece disposed under and connected fixedly to the upper limb part, a lower connecting piece disposed under the upper connecting piece and disposed over and connected fixedly to the lower limb part, and a locking member for locking the upper and lower connecting pieces releaseably on each other. The locking member is operable to permit relative movement of the upper and lower connecting pieces in a transverse direction of the artificial limb in such a manner that the upper and lower connecting pieces are retained on the locking member.

Preferably, the locking member is configured as a vertical lock bolt that is screwed tight to lock the upper and lower connecting pieces on each other and that can be loosened to permit the relative movement between the upper and lower connecting pieces.

### BRIEF DESCRIPTION OF THE DRAWINGS

These and other features and advantages of this invention will become apparent in the following detailed description of the preferred embodiments of this invention, with reference to the accompanying drawings, in which:

FIG. 1 is a fragmentary exploded perspective view of a conventional artificial limb;

FIG. 2 is a fragmentary exploded perspective view of the first preferred embodiment of an artificial limb according to this invention;

FIG. 3 is a perspective view of an upper connecting piece that is inverted to illustrate the structure of a serrated horizontal bottom surface thereof;

FIG. 4 is a fragmentary assembled perspective view of the first preferred embodiment;

FIGS. 5, 6, and 7 are fragmentary schematic sectional views of the first preferred embodiment, illustrating how the position of an upper limb part is adjusted relative to a lower limb part; and

FIG. 8 is a fragmentary exploded perspective view of the second preferred embodiment of an artificial limb according to this invention.

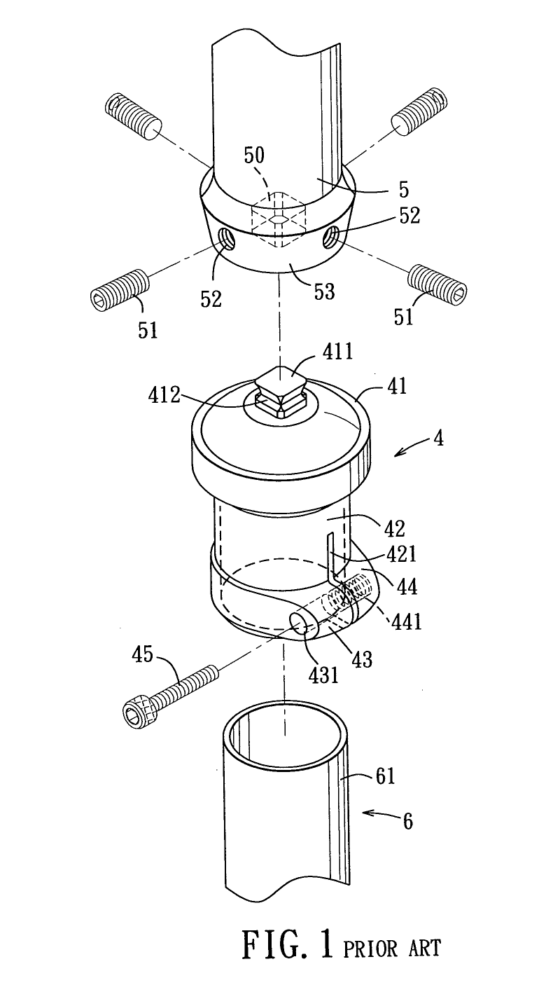

### DETAILED DESCRIPTION OF THE PREFERRED EMBODIMENTS

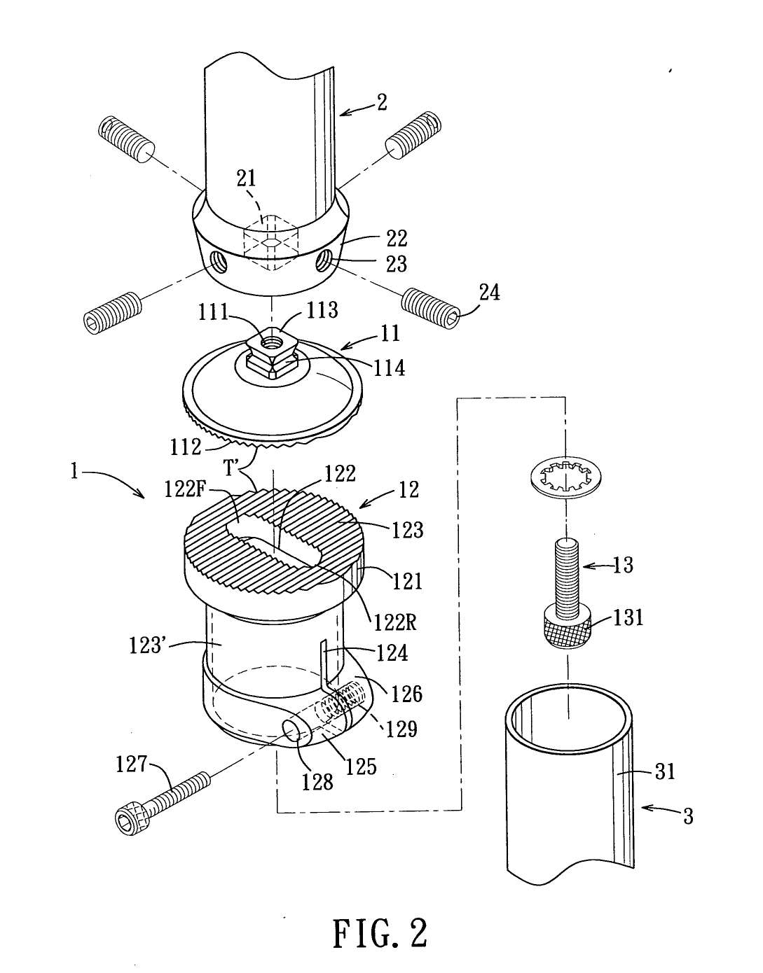

Referring to FIGS. 2, 3, and 4, the first preferred embodiment of an artificial limb according to this invention is shown to include a connecting unit 1, a prosthetic upper limb part 2, and a prosthetic lower limb part 3. The connecting unit 1 is disposed between the upper and lower limb parts 2, 3, and includes an upper connecting piece 11, a lower connecting piece 12, and a locking member 13.

The upper connecting piece 11 is formed with a vertical threaded hole 111 therethrough, and a serrated horizontal bottom surface 112.

The lower connecting piece 12 has a top wall 121 that is formed with a slide slot 122 which extends along a transverse direction (T) (see FIG. 4) of the artificial limb.

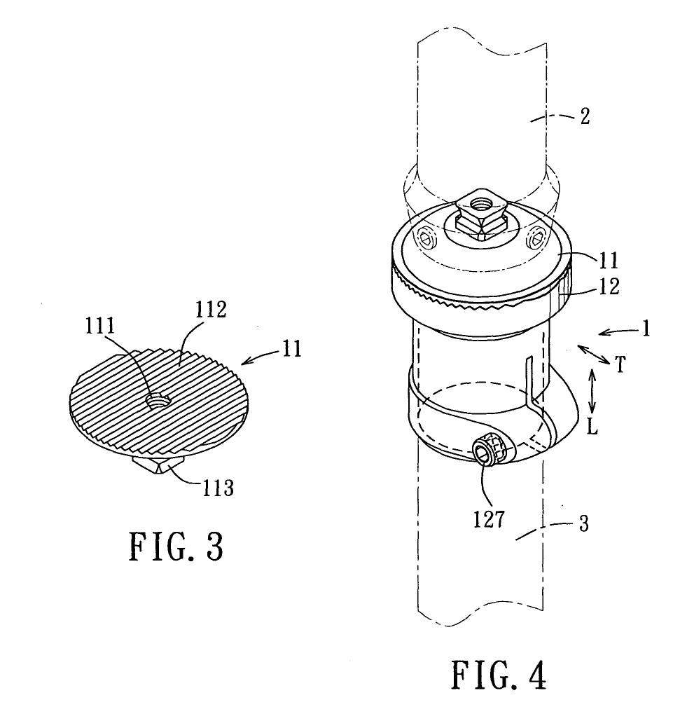

The locking member 13 is configured as a vertical lock bolt that extends along a longitudinal direction (see FIG. 3) of the artificial limb and that extends through the slide slot 122 in the lower connecting piece 12 to engage the threaded hole 111 in the upper connecting piece 11. When the locking member 13 is screwed tight, a head unit 131 of the locking member 13, which consists of an integral head portion of the locking member 13 and a washer that is sleeved around the locking member 13 above the head portion of the locking member 13 and that has an outer diameter greater than the width of the slide slot 122 in the lower connecting piece 12, presses against the top wall 121 of the lower connecting piece 12 so as to lock the lower connecting piece 12 relative to the upper connecting piece 11. When the locking member 13 is loosened, the head unit 131 of the locking member 13 is spaced apart from the top wall 121 of the lower connecting 12 so as to unlock the lower connecting piece 12 from the upper connecting piece 11.

The lower connecting piece 12 has a serrated horizontal top surface 123. Each of the serrated horizontal top and bottom surfaces 123, 112 is formed with a plurality of parallel teeth (T′) (see FIG. 2) that extend along a direction perpendicular to the slide slot 122 in the lower connecting piece 12. When the locking member 13 is screwed tight, the serrated horizontal top and bottom surfaces 123, 112 engage each other to prevent relative movement between the upper and lower connecting pieces 11, 12. When the locking member 13 is loosened, the horizontal top and bottom surfaces 123, 112 are removable from each other to permit relative movement between the upper and lower connecting pieces 11, 12.

The upper connecting piece 11 has an integral tongue 113 that extends upward therefrom and that is formed with an annular groove 114 which extends around the threaded hole 111. The upper limb part 2 includes a bottom surface with a retaining hole 21 for receiving the tongue 113 of the upper connecting piece 11, a surrounding wall 22 defining the retaining hole 21 and having a plurality of bolt holes 23 that are formed therethrough and that are communicated with the retaining hole 21, and a plurality of inclined lock bolts 24 extending respectively through the bolt holes 23 in the surrounding wall 22 and pressing against the tongue 113 of the upper connecting piece 11. The inclined lock bolts 24 engage the annular groove 114 in the tongue 113 of the upper connecting piece 11 so as to lock the upper connecting piece 11 on the upper limb part 2.

The lower connecting piece 12 has a hollow cylindrical lower end portion 123′ that is sleeved around a cylindrical upper end 31 of the lower limb part 3 and that includes a vertical slot 124 with an open lower end, a pair of first and second projections 125, 126, and a horizontal lock bolt 127. The first and second projections 125, 126 are formed integrally with the lower end portion 123′ of the lower connecting piece 12, are located at two sides of the slot 124, and are disposed adjacent to the slot 124. The first projection 125 is formed with a horizontal non-threaded hole 128. The second projection 126 is formed with a horizontal threaded hole 129. The horizontal lock bolt 127 extends through the non-threaded hole 128 in the first projection 126, engages the threaded hole 129 in the second projection 126, is screwed tight to lock the lower limb part 3 on the lower connecting piece 12, and is capable of being loosened to permit removal of the lower limb part 3 from the lower connecting piece 12.

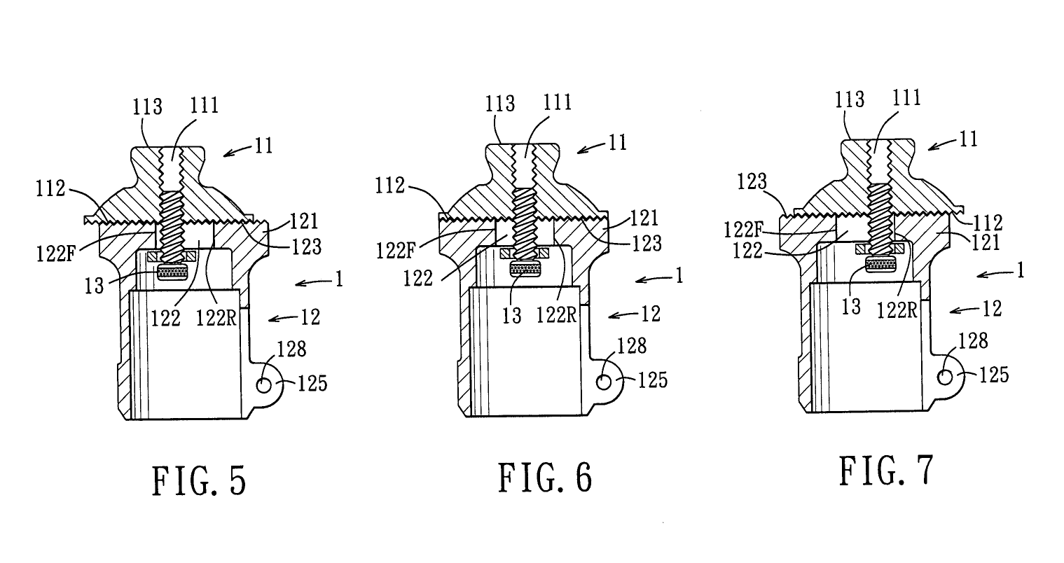

As such, when it is desired to adjust the relative position of the upper and lower limb parts 2, 3, the locking member 13 is somewhat loosened, as shown in FIGS. 5, 6, and 7, so as to move along a transverse direction (T) of the artificial limb between two limit positions shown in FIGS. 5 and 7, where the locking member 13 is disposed respectively at a first end (122F) and a second end (122R) of the slide slot 122. During adjustment, the teeth (T′) of the serrated horizontal bottom and top surfaces 112, 123 can provide an indexing effect.

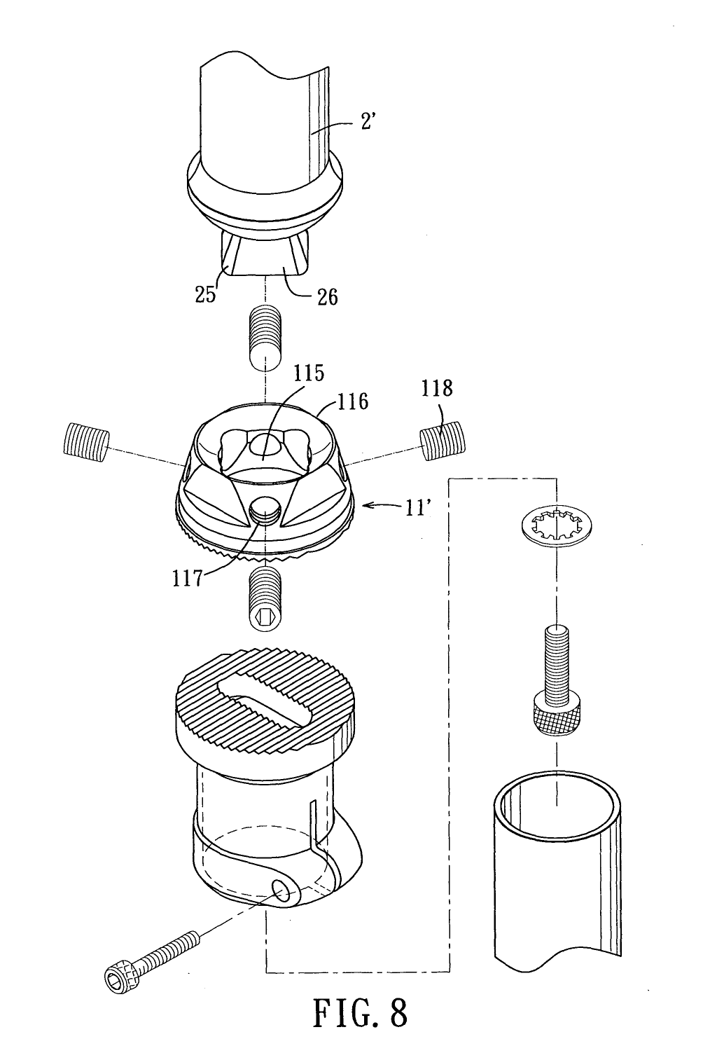

FIG. 8 shows the second preferred embodiment of an artificial limb according to this invention, which is similar to the previous preferred embodiment in construction except for the manner the upper connecting piece 11′ is connected fixedly to the upper limb part 2′. The upper limb part 2′ has a bottom surface that is formed with an integral tongue 25 which has four downwardly and outwardly inclined side surfaces 26. The upper connecting piece 11′ includes a top surface with a retaining hole 115 for receiving the tongue 25 of the upper limb part 2′, a surrounding wall 116 defining the retaining hole 115 and having four bolt holes 117 that are formed therethrough and that are communicated with the retaining hole 115, and four inclined lock bolts 118 extending respectively through the bolt holes 117 in the surrounding wall 116 and pressing against the inclined side surfaces 26 of the tongue 25 of the upper limb part 2′ so as to lock the upper connecting piece 11′ on the upper limb part 2′.

With this invention thus explained, it is apparent the numerous modifications and variations can be made without departing from the scope and spirit of this invention. It is therefore intended that this invention be limited only as indicated by the appended claims.

1. An artificial limb comprising:
a prosthetic upper limb part;
a prosthetic lower limb part disposed under said upper limb part;
an upper connecting piece disposed under and connected fixedly to said upper limb part;
a lower connecting piece disposed under said upper connecting piece and disposed over and connected fixedly to said lower limb part; and
a locking member for locking said upper and lower connecting pieces releaseably on each other, said locking member being operable to permit relative movement of said upper and lower connecting pieces in a transverse direction of said artificial limb in such a manner that said upper and lower connecting pieces are retained on said locking member.

2. The artificial limb as claimed in claim 1, wherein said upper connecting piece is formed with a vertical threaded hole, said lower connecting piece having a top wall that is formed with a slide slot which extends along said transverse direction of said artificial limb, said locking member being configured as a vertical lock bolt that extends along a longitudinal direction of said artificial limb and that extends through said slide slot in said lower connecting piece to engage said threaded hole in said upper connecting piece, said locking member having a head unit that presses against said top wall of said lower connecting piece so as to lock said lower connecting piece relative to said upper connecting piece when said locking member is screwed tight and that is spaced apart from said top wall of said lower connecting piece so as to unlock said lower connecting piece from said upper connecting piece when said locking member is loosened.

3. The artificial limb as claimed in claim 2, wherein said upper connecting piece has a serrated horizontal bottom surface, said lower connecting piece having a serrated horizontal top surface, each of said serrated horizontal top and bottom surfaces being formed with a plurality of parallel teeth that extend along a direction perpendicular to said slide slot, said serrated horizontal top and bottom surfaces engaging each other to prevent relative movement between said upper and lower connecting pieces when said locking member is screwed tight, said horizontal top and bottom surfaces being removable from each other to permit relative movement between said upper and lower connecting pieces when said locking member is loosened.

4. The artificial limb as claimed in claim 2, wherein said upper connecting piece has an integral tongue that extends upward therefrom and that is formed with an annular groove which extends around said threaded hole, said upper limb part including:
a bottom surface with a retaining hole for receiving said tongue of said upper connecting piece;
a surrounding wall defining said retaining hole and having a plurality of bolt holes that are formed therethrough and that are communicated with said retaining hole; and
a plurality of inclined lock bolts extending respectively through said bolt holes in said surrounding wall and pressing against said tongue of said upper connecting piece, said inclined lock bolts engaging said annular groove in said tongue of said upper connecting piece so as to lock said upper connecting piece on said upper limb part.

5. The artificial limb as claimed in claim 2, wherein said upper limb part has a bottom surface that is formed with an integral tongue that has a plurality of downwardly and outwardly inclined side surfaces, said upper connecting piece including:
a top surface with a retaining hole for receiving said tongue of said upper limb part;
a surrounding wall defining said retaining hole and having a plurality of bolt holes that are formed therethrough and that are communicated with said retaining hole; and
a plurality of inclined lock bolts extending respectively through said bolt holes in said surrounding wall and pressing against said inclined side surfaces of said tongue of said upper limb part so as to lock said upper connecting piece on said upper limb part.

6. The artificial limb as claimed in claim 1, wherein said lower limb part has a cylindrical upper end, said lower connecting piece having a hollow cylindrical lower end portion that is sleeved around said cylindrical upper end of said lower limb part and that includes:
a vertical slot with an open lower end;
a pair of first and second projections connected fixedly to said lower end portion of said lower connecting piece and located at two sides of said slot, said first and second projections being disposed adjacent to said slot, said first projection being formed with a horizontal non-threaded hole therethrough, said second projection being formed with a horizontal threaded hole; and
a horizontal lock bolt extending through said non-threaded hole in said first projection and engaging said threaded hole in said second projection, said horizontal lock bolt being screwed tight to lock said lower limb part on said lower connecting piece and being capable of being loosened to permit removal of said lower limb part from said lower connecting piece.
